(19) United States
(12) Patent Application Publication
(10) Pub. No.: US 2014/0088430 A1
(43) Pub. Date: Mar. 27, 2014

(54) ULTRASONIC IMAGE GUIDANCE OF TRANSCUTANEOUS PROCEDURES

(71) Applicant: McKee Dunn POLAND, Andover, MA (US)

(72) Inventor: McKee Dunn POLAND, Andover, MA (US)

(73) Assignee: KONINKLIJKE PHILIPS N.V., Eindhoven (NL)

(21) Appl. No.: 14/022,275

(22) Filed: Sep. 10, 2013

Related U.S. Application Data
(60) Provisional application No. 61/704,806, filed on Sep. 24, 2012.

(51) Int. Cl. A61B 8/08; A61B 8/00

(52) U.S. Cl. CPC A61B 8/0841; A61B 8/4494; A61B 8/085; A61B 8/461
USPC 600/447; 600/461

(57) ABSTRACT

Ultrasonic image guidance of a transcutaneous invasive procedure such as a needle biopsy is conducted by placing an imaging probe above but laterally to one side of a mass to be biopsied. The image region of the probe is then laterally steered in the elevation direction to image the site of the mass to the side of the probe. The mass can then be accessed by needle insertion from immediately above the mass and away from interference with the probe. Preferably the probe has a 2D array transducer so that the image guidance can be viewed in real time three dimensional imaging.

### CROSS-REFERENCE TO PRIOR APPLICATIONS

This application claims the benefit of U.S. Provisional Patent Application No. 61/704,806, filed Sep. 24, 2012, the entire contents of which are incorporated herein by reference.

This invention relates to medical diagnostic ultrasonic imaging and, in particular, to the use of real time ultrasonic imaging to guide the insertion of a transcutaneous surgical device such as a biopsy needle.

Ultrasound guidance for transcutaneous surgical procedures such as the insertion of a biopsy needle is extremely useful and has become the standard of care for some applications. The entry point of the needle into the skin, however, is constrained to positions immediately adjacent to the probe footprint on the skin. When a two dimensional (2D) imaging probe is used, the needle must pass through and align with the plane of the 2D image so it can be visualized as it is guided to the target anatomy. This imposes a further constraint, which is physical alignment of the needle with the image plane. To provide this constraint, needle guides are available which clip onto the body of the probe and constrain the path of the needle to the image plane directly below the probe footprint. Specialized needle insertion probes are known which have a split transducer, allowing the needle to pass through a constraining aperture between the parts of the transducer and into the image plane beneath the probe footprint. When a three dimensional imaging probe is used, a volume beneath the probe footprint can be scanned instead of merely one plane, enabling insertion to be made from multiple positions around the probe footprint, as described in U.S. patent application No. 61/665,476, filed Jun. 28, 2012 (Robinson et al.)

In some cases, the constraint of inserting the needle adjacent to the probe and guiding it to anatomy beneath the probe footprint is a problem due to anatomy which interferes with the restricted path of the needle to the target, or due to the clinician's preference for a steep insertion angle. Incidences of needle insertions actually passing through (and damaging) the lens of the probe substantiate the need for more latitude in needle placement under ultrasound image guidance.

One system which avoids these constraints on needle guidance is an electromagnetic tracking system which tracks the positions of the needle, the body of the patient, and the ultrasound image plane in three dimensional space. Such a system is available from Philip Healthcare of Andover, Mass. and known as the Percunav® system with navigation. Electromagnetic tracking systems are complex, however, requiring the generation of an electromagnetic field around the surgical site, the placement of tracking devices on the needle, the probe, and the patient, and connection of the tracking devices to the tracking system. Many clinicians do not have access to such sophisticated systems and prefer to perform their needle insertions free hand with simply an imaging probe. It is therefore desirable to provide an imaging technique which obviates the aforementioned constraints on needle insertion without the need for complex systems such as electromagnetic navigation systems.

In accordance with the principles of the present invention, an ultrasonic image guidance technique for transcutaneous procedures is described which enables the ultrasound probe to be laterally offset above the surgical site, permitting needle entry to be made at a position laterally offset from the probe footprint such as directly above the surgical site. The surgical site is imaged by placing an ultrasonic imaging probe against the skin of a patient so that ultrasound is acoustically coupled between the probe transducer and the body of the patient. The footprint of the probe contact with the skin is laterally displaced to the side of the surgical site. The image region of the probe is steered laterally to the surgical site which is not beneath the probe footprint on the skin. The needle thus does not need to be guided to a location beneath the probe footprint. The lateral steering of the image region can be attained in several ways, including lateral electronic beam steering, scanning from the side of a curved transducer array, or use of a probe with an angled lens or standoff. The inventive technique provides a full image of the laterally adjacent target area for the procedure and enables needle insertion away from the body of the probe. The inventive technique may be performed in both 2D and 3D imaging modes.

In the drawings:

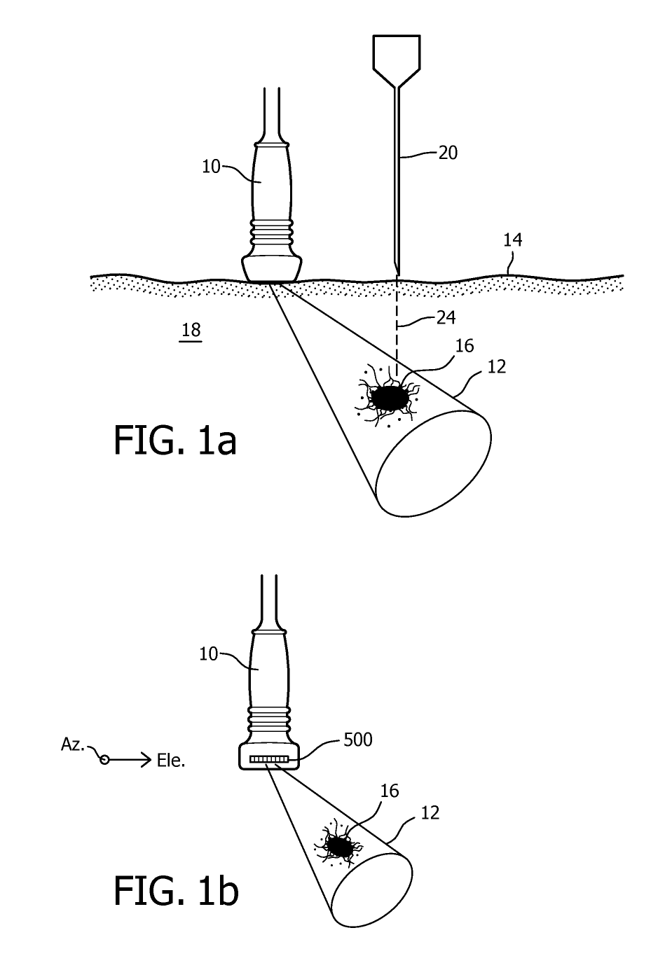

FIGS. 1a and 1b illustrate ultrasonic image guided transcutaneous needle insertion in accordance with a first example of the present invention which employs an ultrasound probe with electronic beam steering.

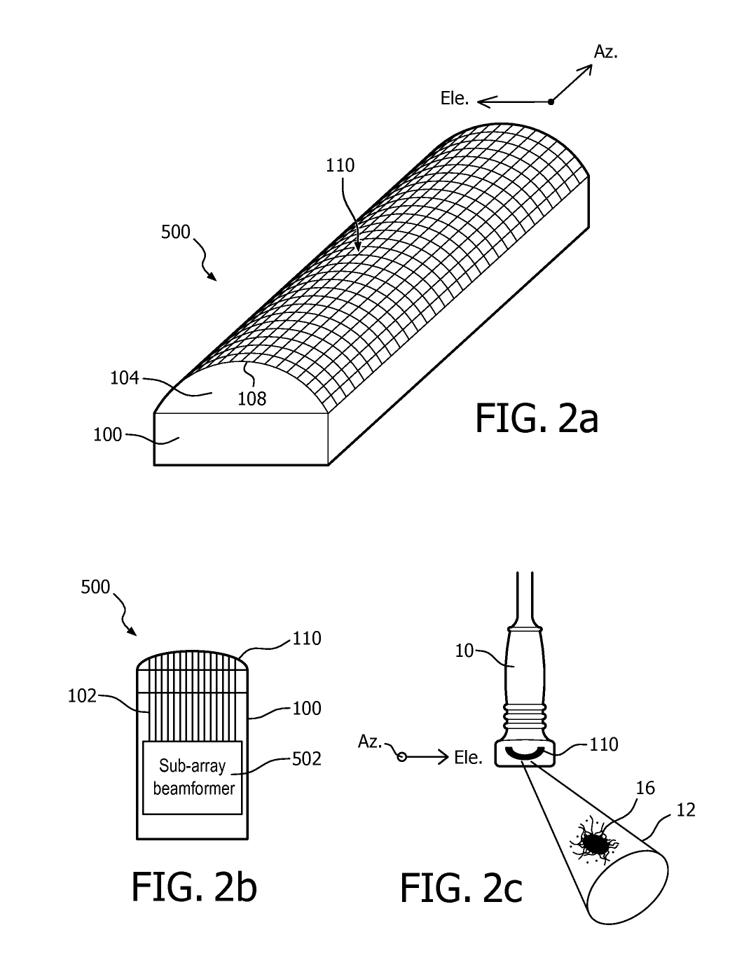

FIGS. 2a, 2b and 2c illustrate ultrasonic image guided transcutaneous needle insertion in accordance with a second example of the present invention which employs an ultrasound probe with a curved array transducer.

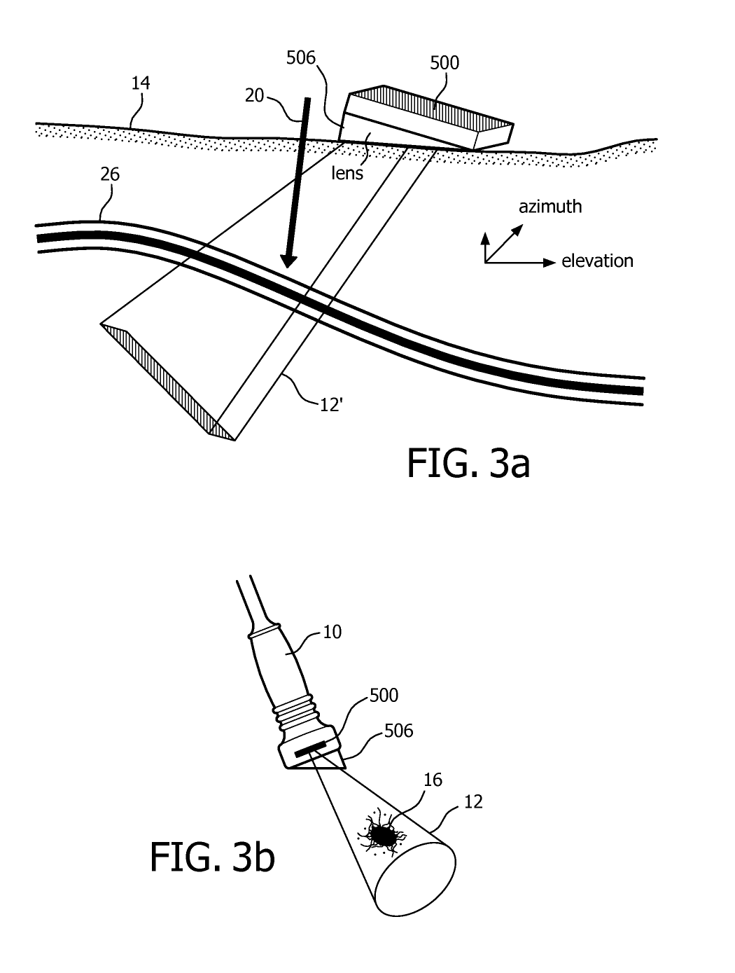

FIGS. 3a and 3b illustrate ultrasonic image guided transcutaneous needle insertion in accordance with a third example of the present invention which employs an ultrasound probe with a tapered or angled lens or standoff.

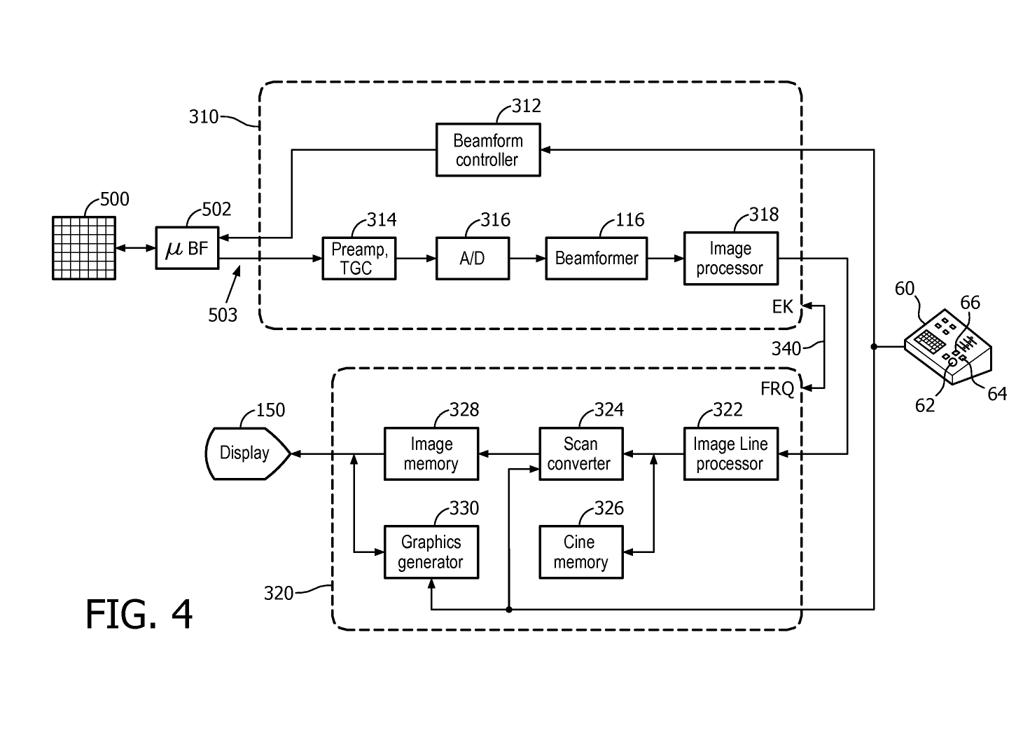

FIG. 4 illustrates an ultrasonic diagnostic imaging system suitable for use in the conduct of the inventive technique.

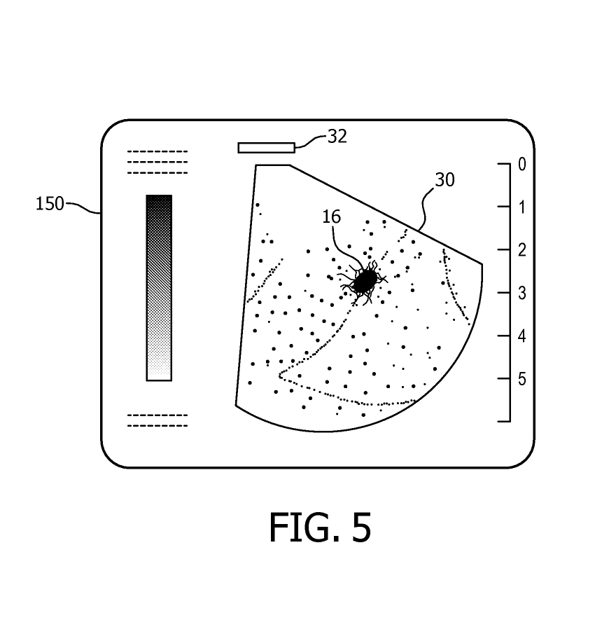

FIG. 5 illustrates an ultrasound display of a surgical site in accordance with the principles of the present invention with a probe footprint graphic that helps orient a clinician to the location of the surgical site in relation to the position of the ultrasound probe.

Referring to FIGS. 1a and 1b, a first example of ultrasound-guided needle insertion in accordance with the present invention is shown. In this example a biopsy needle 20 is to be inserted through the skin line 14 and into the body 18 of a patient to access a mass 16 for biopsy. The mass 16 is imaged by an ultrasound probe 10 acoustically coupled to the body of the patient so that the clinician can guide the needle 20 to the mass 16 by reference to an ultrasound image of both the approaching needle and the mass. Acoustic coupling is generally enhanced by a coating of acoustic gel on the skin where the probe is to be placed. The surgical site is not directly below the footprint of the probe 10 but is laterally off to one side as shown in FIG. 1a. The mass 16 is imaged by steering the image region of the probe laterally as shown in the drawing. A probe with a mechanically steered transducer array may be used, but preferably the probe has an electronically steered array to steer the beams to and from the probe. The mass can be imaged by a probe with a one dimensional (1D) array but preferably is imaged three dimensionally by a probe with an electronically steered 2D array transducer. The extent of a 2D array of transducer elements is in the azimuth and elevation directions and the beam steering is generally done in the elevation direction. Three dimensional imaging is preferred because it is then not necessary to hold the probe so that the mass 16 is continually in a single image plane. Instead, the mass 16 can be imaged in the center of a volumetric region 12 imaged by the probe 10. If there is unintended movement of the patient or the probe, the mass can generally be kept constantly within view in the imaged volume. The volumetric region can be of any desired shape depending upon the pattern of beam steering. In the example of FIG. 1a the volumetric region is conical in shape. Pyramidal or other volumetric region shapes may also be used.

With the probe 10 positioned on and acoustically coupled to the skin 14 to the side of the location of the mass 16, the clinician can readily access the mass 16 by the most direct path through the body such as from directly above as indicated by the dashed line insertion path 24. The clinician has ample space around the needle insertion point to position and manipulate the needle for accurate insertion because the probe 10 is off to the side of the insertion site. There is no possibility of damaging the probe 10 with the needle.

FIG. 1b illustrates the relationship between the transducer array 500 of the probe and the volume 12 being imaged. The elevation direction (Ele.), the direction to the side in which the image region is steered, is in the plane of the drawing sheet. The azimuth direction (Az.) is normal to the drawing sheet. The depth direction is the third dimension into the body of the patient.

FIGS. 2a, 2b and 2c illustrate a second implementation of the ultrasound-guided needle insertion technique of the present invention, this time using a curved array transducer 500 as shown in the perspective view of FIG. 2a. The transducer array is a curved array of transducer elements 110. A preferred shape for the curved array is a two dimensional array curved in a semi-cylindrical shape as shown in FIG. 2a. The elements 110 of the array are arranged in linear rows in the azimuth direction and are curved in the elevation direction as indicated by the curved array surface 108. The array of transducer elements 110 is mounted on a support substrate 140 which is generally heat conductive. Behind the substrate 104 is an acoustically attenuating backing block 100. Located in the backing block 100 is a sub-array beamformer with conductors 102 connected to the elements of the array as shown in FIG. 2b. The conductors 102 may be directly connected through the support substrate 104 or by an interposer which matches different pitches of the array elements and connections to the sub-array beamformer. Suitable semi-cylindrical arrays are described in U.S. Pat. No. 7,927,280 (Davidsen), U.S. Pat. No. 7,741,756 (Sudol) and U.S. Pat. No. 8,161,817 (Robinson et al.) Suitable sub-array beamformers, also referred to in the art as microbeamformers, are described in these patents and also in U.S. Pat. No. 5,997,479 (Savord et al.) and U.S. Pat. No. 6,013,032 (Savord).

FIG. 2c illustrates the relationship between the curved transducer array 500 of the probe 10 and the volume 12 being imaged. The azimuth and elevation directions are the same as in FIG. 1b. Since the 2D array of elements 110 is curved in the elevation dimension much of the lateral beam steering is provided by the physical curvature of the array. The beams of the volume 12 are more normal to the surface 108 of the array than in FIG. 1a, providing greater sensitivity for received echo signals than is the case with steeper angled beam steering. The physical beam steering also reduces the time and range of delays needed from the sub-array beamformer 502 for proper beam formation.

FIGS. 3a and 3b illustrate a third example of the ultrasound-guided needle insertion technique of the present invention, this time using a planar array transducer 500 with a tapered lens or standoff 506. The lens or standoff may be a permanently attached lens which provides a probe 10 dedicated for the procedure, or may be an attachable and removable standoff which adapts a conventional probe configuration to one with the desired angle in relation to the skin surface 14. The taper of the lens 506 is in the elevation direction which causes the array transducer 500 to be angled non-orthogonally in relation to the surface of the skin. This means that beams passing to and from the array 500 are tilted to one elevational side of the transducer as shown in the drawing. Similar to the probe of FIGS. 2a-2c, the tilt provided by the tapered lens provides physical steering of the beams, in this case to the left of the probe in the elevation dimension as shown in FIG. 3a. In this example the array transducer 500 is a 2D array which scans a pyramidal volumetric region 12′ containing the surgical site. The anatomy being accessed in this example is a nerve bundle 26 which passes through the volumetric region and is accessed by needle 20 on the left side of the probe. FIG. 3b illustrates the relationship between the tilted transducer array 500 of the probe 10 and the volume 12′ being imaged which is provided by the tapered lens 506. Similar to the probe of FIGS. 2a-2c, the beams of the scanned volume are more nearly orthogonal to the surface of the planar array 500 than is the case with the probe of FIGS. 1a-1b, with the same benefits of sensitivity and delay requirements.

FIG. 4 illustrates an ultrasound system in block diagram form which is suitable for use in the practice of the present invention. The probe 10 includes a two-dimensional array transducer 500 and a sub-array processor or micro-beamformer 502. The microbeamformer contains circuitry which control the signals applied to groups of elements (“patches”) of the array transducer 500 and does some processing of the echo signals received by elements of each group. Microbeamforming in the probe advantageously reduces the number of conductors in the cable 503 between the probe and the ultrasound system and is described in the aforementioned Savord et al. patent and in U.S. Pat. No. 6,436,048 (Pesque). The probe 10 is coupled to the scanner 310 of the ultrasound system. The scanner includes a beamformer controller 312 which is responsive to a user control 60 and provides control signals to the microbeamformer 502 instructing the probe as to the timing, frequency, direction and focusing of transmit beams. In the practice of the present invention this control steers the scanned plane or volume to the surgical site to the side of the probe. In this implementation a control button 66 is used to select image steering and a trackball 62 is manipulated to steer the image in the desired direction so that the surgical site is in view. Once the image plane or volume is desirably oriented in this manner, a button 64 is pressed to select and fix the desired image orientation. The beamformer controller also controls the beamforming of received echo signals by its coupling to analog-to-digital (A/D) converters 316 and a beamformer 116. Echo signals received by the probe are amplified by preamplifier and TGC (time gain control) circuitry 314 in the scanner, then digitized by the A/D converters 316. The digitized, partially beamformed echo signals are then fully formed into beams by a system beamformer 116. The echo signals are processed by an image processor 318 which performs digital filtering, B mode detection, and Doppler processing, and can also perform other signal processing such as harmonic separation, speckle reduction through frequency compounding, and other desired image processing. B mode echoes from each received scanline are processed by amplitude detection in the image processor 318, and Doppler echo ensembles are Doppler processed in the image processor for the production of display signals depicting flow or tissue motion. The processed B mode and/or Doppler signals are then coupled to the display subsystem 320 for display.

The display subsystem 320 processes the echo signals for display in the desired image format. The echo signals are processed by an image line processor 322, which is capable of sampling the echo signals, splicing segments of beams into complete line signals, and averaging line signals for signal-to-noise improvement or flow persistence. The image lines are scan converted into the desired image format by a scan converter 324 which performs R-theta conversion and volume rendering of 3D images as is known in the art. The image is then stored in an image memory 328 from which it can be displayed on a display 150. The image in memory is also overlayed with graphics to be displayed with the image, which are generated by a graphics generator 330 which is responsive to the user control for the input of patient identifying information or the movement of cursors, for example. Individual images or image sequences can be stored in a cine memory 326 during capture of image loops.

For real-time volumetric imaging the display subsystem 320 also includes the 3D image rendering processor referred to above (not separately shown) which receives image lines from the image line processor 322 for the rendering of a real-time three dimensional image which is displayed on the display 150.

FIG. 5 illustrates an ultrasound image display 150 which displays real time images 30 of a surgical site, a mass 16 which is to be biopsied. Patient data and other information is displayed above and below the grayscale or color bar to the left of the ultrasound images 30 by means of the graphics generator 330. In accordance with a further aspect of the present invention the ultrasound images are displayed in an orientation which shows the relation between the footprint of the probe and the image region. A graphic 32 is displayed above the images 30, which identifies the probe footprint where the probe contacts the skin of the patient. The graphic is also produced by the graphics generator 330. The graphic and image orientations clearly depict the lateral offset of the location of the mass 16 to the right of the probe, providing guidance to the clinician as to the distance to the right of the probe where the needle can be inserted to directly access the mass 16. The clinician can then begin to insert the needle above the mass 16 and as the needle enters the plane or volume of the image (preferably a 3D volume image), the needle will be seen as it enters the imaged region and can be guided to access the mass 16 to be biopsied.

What is claimed is:

1. A method for ultrasonically guiding a transcutaneous surgical procedure by real time ultrasonic imaging comprising:
placing an ultrasonic imaging probe in acoustic coupling contact with the skin of a subject so that the footprint of the probe contact with the skin is laterally displaced from a location on the skin directly above a surgical site in the subject;
steering an image region of the probe laterally to image the surgical site to one side of the probe footprint;
inserting an invasive device into the skin of the subject at a location to the side of the probe footprint; and
observing the relative positions of the invasive device and the surgical site in real time ultrasound images as the invasive device approaches the surgical site.

2. The method of claim 1, wherein steering an image region further comprises electronically steering beams from an array transducer in the probe.

3. The method of claim 2, wherein electronically steering beams from an array transducer further comprises electronically steering beams to scan a volumetric region in the subject which includes the surgical site.

4. The method of claim 3, wherein electronically steering beams further comprises electronically steering beams from a two dimensional array transducer.

5. The method of claim 1, wherein steering an image region further comprises transmitting and receiving beams from a side of a curved array transducer in the probe.

6. The method of claim 5, wherein steering an image region further comprises scanning a volumetric region in the subject which includes the surgical site.

7. The method of claim 6, wherein steering an image region further comprises transmitting and receiving beams from a side of a curved two dimensional array transducer.

8. The method of claim 1, wherein steering an image region further comprises utilizing a tapered lens or standoff between a transducer array in the probe and the skin of the subject.

9. The method of claim 8, wherein steering an image region further comprises scanning a volumetric region in the subject which includes the surgical site.

10. The method of claim 9, wherein steering an image region further comprises transmitting and receiving beams through the tapered lens or standoff with a two dimensional array transducer.

11. The method of claim 4, wherein the two dimensional array transducer exhibits an azimuth dimension and an elevation dimension,
wherein electronically steering beams further comprises steering beams laterally in the elevation direction.

12. The method of claim 7, wherein the two dimensional array transducer is curved in an elevation dimension,
wherein transmitting and receiving beams further comprises transmitting and receiving beams laterally in the elevation direction.

13. The method of claim 10, wherein the two dimensional array transducer has an azimuth direction and an elevation direction;
wherein the tapered lens or standoff with the two dimensional array transducer is tapered in the elevation dimension,
wherein transmitting and receiving beams through the tapered lens or standoff further comprises transmitting and receiving beams laterally in the elevation direction.

14. The method of claim 1, further comprising displaying the real time images on a display together with a graphic indicating the position of the probe footprint in relation to the location of the steered image region.

15. The method of claim 14, wherein the graphic is displayed above the real time images and the real time images display the surgical site below and laterally to one side of the graphic.
